(19) United States
(12) Patent Application Publication
(10) Pub. No.: US 2012/0178136 A1
(43) Pub. Date: Jul. 12, 2012

(54) FERMENTATION PROCESS FOR PRODUCING GLYCOLIC ACID

(75) Inventors: Wanda Dischert, Vic-Le-Comte (FR); Cédric Colomb, Clermont-Ferrand (FR); Philippe Soucaille, Deyme (FR)

(73) Assignee: ROQUETTE FRERES, LESTREM (FR)

(21) Appl. No.: 13/497,912

(22) PCT Filed: Sep. 23, 2010
(86) PCT No.: PCT/EP10/64058

§ 371 Date: Mar. 23, 2012

Related U.S. Application Data
(60) Provisional application No. 61/245,716, filed on Sep. 25, 2009.

(30) Foreign Application Priority Data
Sep. 25, 2009 (EP) 09171297.6

(51) Int. Cl. C12P 7/42

(52) U.S. Cl. 435/146

(57) ABSTRACT

The present invention relates to a process of fermentation for producing glycolic acid under specific pH conditions with an increase of the pH during fermentation. The invention relates more particularly to a method for producing glycolic acid by fermentation, which comprises culturing a microorganism having glycolic acid producing ability in an appropriate culture medium with a carbon source, and under specific pH conditions with an increase of the pH during fermentation, and recovery of glycolic acid from the culture medium.

### FIELD OF THE INVENTION

The present invention relates to a process of fermentation for producing glycolic acid under specific pH conditions with an increase of the pH during fermentation.

### BACKGROUND OF THE INVENTION

Glycolic Acid (HOCH₂COOH), or glycolate, is the simplest member of the alpha-hydroxy acid family of carboxylic acids. Glycolic acid has dual functionality with both alcohol and moderately strong acid functional groups on a very small molecule. Its properties make it ideal for a broad spectrum of consumer and industrial applications, including use in water well rehabilitation, the leather industry, the oil and gas industry, the laundry and textile industry, and as a component in personal care products.

Glycolic Acid can also be used to produce a variety of polymeric materials, including thermoplastic resins comprising polyglycolic acid. Resins comprising polyglycolic acid have excellent gas barrier properties, and such thermoplastic resins comprising polyglycolic acid may be used to make packaging materials having the same properties (e.g., beverage containers, etc.). The polyester polymers gradually hydrolyze in aqueous environments at controllable rates. This property makes them useful in biomedical applications such as dissolvable sutures and in applications where a controlled release of acid is needed to reduce pH. Currently more than 15,000 tons of glycolic acid are consumed annually in the United states.

Although Glycolic Acid occurs naturally as a trace component in sugarcane, beets, grapes and fruits, it is mainly synthetically produced. Other technologies to produce Glycolic Acid are described in the literature or in patent applications. For instance, Mitsui Chemincals, Inc. has described a method for producing the said hydroxycarboxylic acid from aliphatic polyhydric alcohol having a hydroxyl group at the end by using a microorganism (EP 2 025 759 A1 and EP 2 025 760 A1). This method is a bioconversion as the one described by Michihiko Kataoka in its paper on the production of glycolic acid using ethylene glycol-oxidizing microorganisms (Biosci. Biotechnol. Biochem., 2001). Glycolic acid is also produced by bioconversion from glycolonitrile using mutant nitrilases with improved nitrilase activity and that technique was disclosed by Dupont de Nemours and Co in WO2006/069110. Methods for producing Glycolic Acid by fermentation from renewable resources using other bacterial strains are disclosed in patent applications from Metabolic Explorer (WO 2007/141316 and U.S. 61/162,712 and EP 09155971.6 filed on 24 Mar. 2009).

Several other hydroxycarboxylic acids, including citric acid, lactic acid and gluconic acid are produced by fermentation processes as are other acids such as succinic acid. The methods suitable for the maintenance and growth of bacterial cells used and described for these productions make usually reference to the Manual of Method of General Bacteriology, Eds P. Gerhard et al., American Society for Microbiology Washington D.C. (1981) and to A Textbook of Industrial Microbiology, 2^{nd} ed. (1989) Sinauer associates, Sunderland. Md.

A common technique used to produce organic acids is to maintain the pH constant in a desired region by adding an alkali material during the fermentation process as a buffering salt to avoid too acidic conditions detrimental to the microorganism activity when pH values for fermentation with good productivity range from about 5.0 to about 7.0.

The inventors found that applying a shift of the pH rather than a simple buffering of the culture medium induces changes of the metabolism of the microorganism and enhances the yield of production. Increasing the pH reduces the flux towards the biomass without stopping the production of the organic acid resulting in an increased yield of glycolic acid.

### SUMMARY OF THE INVENTION

The present invention concerns a method for producing glycolic acid by fermentation, which comprises culturing a microorganism having glycolic acid producing ability in an appropriate culture medium with a carbon source and recovering the glycolic acid from the culture medium, wherein the culture of the microorganism comprises the following steps:

a) culturing the microorganism at a first pH below 7,
b) increasing the pH of the culture medium to a pH above 7,
c) culturing the microorganism in the culture medium having the increased pH above 7.

Advantageously, the pH is increased at a specific moment of the fermentation according to identified parameters of the fermentation process related to the growth of the strain, particularly the carbon source consumption of the strain and/or the production of glycolic acid.

The pH increase can be made at any rate allowing increase of yields determined by usual experimental procedures. Generally, the step of the pH increase (step b) occurs in about 4% of the total duration of fermentation. The duration of step b) is generally less than 2 hours for a total fermentation time of 50 hours.

The microorganism is advantageously a microorganism selected for having an ability to produce glycolic acid with high yield, more particularly genetically modified for producing glycolic acid with improved yield.

### DETAILED DESCRIPTION OF THE INVENTION

In the present application, terms are employed with their usual meaning, except when specified otherwise.

Microorganism

A “microorganism” means all kind of unicellular organisms, including procaryotic organisms like bacteria, and eucaryotic organisms like yeasts. Preferentially, the microorganism is selected among Enterobacteriaceae, Bacillaceae, Streptomycetaceae and Corynebacteriaceae. More preferentially, the microorganism is a species of Escherichia, Klebsiella, Pantoea, Salmonella or Corynebacterium. Even more preferentially, the microorganism is Escherichia coli.

As used herein, the term “modified microorganism” or “modified” or “recombinant” refer to a host cell that has a modification of its genome, e.g., as by addition of nucleic acid not naturally occurring in the organism or by a modification of nucleic acid naturally occurring in the host cell.

A “microorganism having glycolic acid producing ability” means a microorganism having the ability, when grown under suitable conditions, to produce and accumulate glycolic acid. Preferably, these microorganisms can produce more than 30 g of glycolic acid per L of culture medium, preferably more than 40 g/L and most preferably more than 50 g/L with a yield of production above 0.3 g of glycolic acid per g of carbon source, generally between 0.3 g/g and 0.5 g/g.

Said microorganisms are preferably modified for producing glycolic acid with improved yields. Said modifications are known in the art and include adaptation to a culture medium as well as genetic modification by attenuating and/or deleting and/or replacing and/or overexpressing genes to favour a metabolic pathway for the production of glycolic acid.

Genetically modified strains are known in the art such as strains disclosed in WO2007/141316 and in patent applications U.S. 61/162,712 and EP 09155971.6 filed Mar. 24, 2009 and entitled “Method for producing high amount of glycolic acid by fermentation” incorporated herein by reference.

In particular embodiments, the microorganisms are modified for producing glycolic acid and comprise at least one of the following modifications:
attenuation of the genes ldhA and/or mgsA
attenuation of the gene arcA
attenuation of the membrane import of glycolate (attenuation of glcA, lldP, and yjcG)
attenuation of the conversion of glyoxylate to products other than glycolate (attenuation of aceB, glcB, gcl, eda)
is unable to substantially metabolize glycolate (attenuation of glcDEF, aldA)
increase of the glyoxylate pathway flux (attenuation of icd, aceK, pta, ack, poxB, iclR or fadR, and/or overexpression of aceA)
increase of the conversion of glyoxylate to glycolate (overexpression of ycdW)
increase of the availability of NADPH (attenuation of pgi, udhA, eddy.
attenuation of the gene aceK
and combinations thereof.

Culture Medium and Carbon Source

An “appropriate culture medium” means a medium of known molecular composition adapted to the growth of the micro-organism. In particular, said medium contains at least a source of phosphorus and a source of nitrogen. Said appropriate medium is for example a mineral culture medium of known set composition adapted to the bacteria used, containing at least one carbon source. Said appropriate medium may also designate any liquid comprising a source of nitrogen and/or a source of phosphorus, said liquid being added and/or mixed to the source of sucrose. In particular, the mineral growth medium for Enterobacteriaceae can thus be of identical or similar composition to M9 medium (Anderson, 1946), M63 medium (Miller, 1992) or a medium such as defined by Schaefer et al. (1999), and in particular the minimum culture medium named MML11AG1 100 described in the examples in table 1.

The carbon source ‘glucose’ can be replaced in this medium by any other carbon source, in particular by sucrose or any sucrose-containing carbon source such as sugarcane juice or sugar beet juice.

A “carbon source” or “carbon substrate” means any carbon source capable of being metabolized by a microorganism wherein the substrate contains at least one carbon atom.

Preferably, the carbon source is selected among the group consisting of glucose, sucrose, monosaccharides (such as fructose, mannose, xylose, arabinose) or oligosaccharides (such as galactose, cellobiose . . . ), polysaccharides (such as cellulose), starch or its derivatives, glycerol and mixtures thereof. An especially preferred carbon source is glucose. Another preferred carbon source is sucrose.

Indeed the microorganisms of the present invention can be modified to be able to grow on specific carbon sources, when the non modified microorganism cannot grow on the same source of carbon, or grow at to low rates. These modifications may be necessary when the source of carbon is a byproduct of biomass degradation such as by-products of sugarcane including; filter cake from clarification of raw juice and different kind of molasses.

Modifying microorganisms to allow their growth on specific sources of carbon is known in the art, such as disclosed in the patent application PCT/EP2008/065131 filed on November the 7^{th} 2008 and in the literature for E. coli K12 with the papers of Schmid et al, 1982, Jahreis et al., 2002, Tsunekawa et al., 1992, Penfold and Macaskie, 2004 incorporated herein by reference.

Culture Conditions

Except for the pH increase, the culture conditions are usual conditions known for culturing microorganisms in fermentative methods.

According to the invention the terms ‘cultivating’, ‘culture’, ‘growth’ and ‘fermentation’ are used interchangeably to denote the growth of bacteria in an appropriate growth medium containing a simple carbon source wherein the carbon source is used both for the growth of the strain and for the production of the desired product, glycolic acid.

In the fermentative process of the invention, the source of carbon is used for:
biomass production: growth of the microorganism by converting inter alia the carbon source of the medium, and,
glycolic acid production: transformation of the same carbon source into glycolic acid by the same biomass.

The two steps might be concomitant and the transformation of the source of carbon by the microorganism to grow results in the glycolic acid secretion in the medium, since the microorganism comprises a metabolic pathway allowing such conversion.

Fermentation is a classical process that can be performed under aerobic, microaerobic or anaerobic conditions.

In the invention, the fermentation is done according to a discontinuous fed-batch mode.

This refers to a type of fed-batch in which supplementary growth medium is added during the fermentation, but no culture is removed until the end of the batch. The process comprises two steps; the first one which is the pre culture in MML8AG1 100 (see composition in examples) in Erlenmeyer flask, and the second one which is the culture in MML11AG1 100 medium in fermenter vessel. Each pulse of fed contains growth medium with 20 g/L of glucose, oligo-elements and appropriate antibiotics.

The recovery of the glycolic acid from the culture medium can be made at any time during the fermentation process: during any one of steps a, b or c, or at the end of the culture.

According to the invention, the pH of the culture medium at the start is above pH 6 (step a), preferably ranging from 6 to 7, more preferably from 6.5 to 7.

The pH of the medium is usually adjusted with a base solution of sodium hydroxide (2,5% w/w) and ammonium hydroxide (7,5% w/w) until start of the pH increase (step b).

The pH of the culture medium is increased above pH 7 (step c), preferably ranging from 7 to 8, more preferably from 7.1 to 7.5.

In a preferred embodiment of the invention, the pH of the culture medium is controlled during the fermentation: at the start of the culture the pH is above pH 6, and at a specific moment of the fermentation, the pH is switched to reach a pH below 8 at the end of the culture.

In another preferred embodiment of the invention, the pH of the culture medium is controlled during the fermentation: in step a) the pH of the culture medium is ranging from 6 to 7, preferably from 6.5 to 7.

In another preferred embodiment of the invention, the pH of the culture medium is controlled during the fermentation: in step c) the pH of the culture medium is ranging from 7 to 8, preferably from 7.1 to 7.5.

The interval between the pH in step a) and the pH in step c) is at least of 0.2, preferentially at least of 0.3, more preferentially at least of 0.5.

Nowadays, the fermentation systems used to carry cultures allow an accurate pH regulation. Indeed, Multifors or Sixfors systems from Infors company present an accuracy of 0.1 pH unit and Biolaffite system from Pierre Guerin company allows an accurate regulation of the pH with a precision of 0.01 pH unit.

Advantageously, the pH is increased at a specific moment of the fermentation according to identified parameters of the fermentation process related to the growth of the strain, particularly the carbon source consumption of the strain in the culture medium and/or the production of glycolic acid in the culture medium.

In particular, the step b) is initiated when, in step a), at least one of the following fact is observed:
the carbon source consumption of the strain is superior to 60 g/L, preferably above 80 g/L, more preferably above 100 g/L, and/or
the production of glycolic acid is superior to 20 g/L preferably above 25 g/L.

The pH is generally increased when the carbon source consumption of the strain is ranging from 60 g/L to 160 g/L, preferably from 80 g/L to 140 g/L, more preferably from 100 g/L to 120 g/L.

The pH may also be increased when the production of glycolic acid reaches a glycolic acid concentration is generally ranging from 25 g/L to 50 g/L, preferably from 25 g/L to 40 g/L, more preferably from 25 g/L to 35 g/L.

The “carbon source consumption” values given above are established for glucose. The skilled person will however be in a position to determine the most appropriate consumption values for other carbon sources by simple routine experimentation, such as defining a correlation between production of glycolic acid and carbon source consumption.

In step b), the pH is increased by addition of a base, preferably selected among organic and inorganic bases, including NaOH, NH₄OH, Mg(OH)₂, Ca(OH)₂ and mixtures thereof. The base is preferably in a liquid form, although the person skilled in the art of fermentative production may choose the most appropriate way to increase the pH depending among other factors on the size of the tank and on the system, used for the fermentation by simple experimentation.

In a particular embodiment, the culture medium is lacking ammonium cations and the base is preferably not an ammonium base to create starvation conditions.

The pH increase can be made at any rate allowing increase of the yield determined by usual experimental procedures. Generally, the pH increase occurs in about 4% of the total duration of the fermentation. It is generally less than about 2 hours for a fermentation time of 50 hours.

Indeed, the skilled artisan can decide the most appropriate rate for increasing of the pH, according to specific culture conditions and/or the technical feasibility of an industrial process. In some cases, the operating conditions may impose a faster increase of the pH. However, such fast increase would not impact substantially the yield improvement when timely done.

Recovery

The recovery of the glycolic acid from the culture medium can be made at any time during the fermentation process: during any one of steps a, b or c, or at the end of the culture.

Recovery of the glycolic acid is made by a step of concentration of glycolate in the bacteria or in the medium, and isolation of glycolic acid from the fermentation broth and/or the biomass, optionally remaining in portions or in the total amount (0-100%) in the end product from the fermentation culture.

In a particular embodiment, the process comprises a step of recovery of the glycolic acid produced through a step of polymerization to at least glycolic acid dimers and then recovery of glycolic acid by depolymerisation from glycolic acid dimers, oligomers and/or polymers.

Polymerization methods by two different chemical routes are known in the art; including ring-opening polymerization of cyclic diesters in three steps: (i) polycondensation of α-hydroxycarboxylic acids, (ii) the synthesis of cyclic diesters by a thermal unzipping reaction and (iii) ring-opening polymerization of the cyclic diester (Preparative Methods of Polymer Chemistry 2^{nd} edition, Interscience Publishers Inc, New York 1963, Sorensen, W. R. & Campbell, T. W.; Controlled Ring-opening Polymerization of Lactide and Glycolide. Chem. Rev. 104, 6147-6176 (2004), Dechy-Cabaret, O. et al.). Alternatively, polymerization can be achieved by direct polycondensation of glycolic acid. (Synthesis of polylactides with different molecular weights. Biomaterials 18, 1503-1508 (1997), Hyon, S. -H. et al.) which content is incorporated herein by reference.

### EXAMPLES

The strain genetically engineered to produce glycolic acid from glucose as a carbon source is disclosed in patents WO 2007/141316 A, U.S. 61/162,712 and EP 09155971, 6. The strain used herein in the example is named AG0662F04c01 (MG1655 Ptrc50-RBSB-TTG-icd::Cm ΔaceB Δgcl ΔglcDEFGB ΔaldA ΔiclR Δedd+eda ΔpoxB ΔackA+pta (pME101-ycdW-TT7-PaceA-aceA-TT01).

The two examples show how specific modifications of the pH during the fermentation improve the glycolic acid (GA) production performances of the strain.

### Example 1

### Process of Fermentation for Producing Glycolic Acid Under Specific pH Conditions

### Increase of the pH During the Culture and Impact of the Final pH Value

In this example, the strain AG0662F04c01 was cultivated in different conditions of final pH value, comprised between pH 6.7 to pH 7.6.

Cultures were usually carried out in parallel for each run. A unique preculture was carried out in three 2 l baffled Erlenmeyer flask filled with 220 ml of synthetic medium MML8AG1_{—}100 (Table 1) supplemented with 40 g/l of MOPS, 10 g/l of glucose and 10% of LB medium, at 30° C. during 3 days. These precultures were then concentrated by centrifugation (4000 g, 5 min at 25° C.) in order to recover 20 ml of broth at an optical density of about 22 for each. These concentrated precultures were used to inoculate the cultures.

Cultures were grown in 700 mL working volume vessels assembled on a Multifors System (Multifors Multiple Fermenter System, Infors). Each vessel was filled up with 200 ml of synthetic medium MML11AG1_{—}100 (Table 2) supplemented with 20 g/l of glucose and 50 mg/l of spectinomycin. Each fermenter was inoculated at an initial optical density of about 2.

| | Concentration | | Concentration |
|---|---|---|---|
| Constituent | (g/l) | Constituent | (g/l) |
| Citric acid | 6.00 | Citric acid | 3.00 |
| MgSO₄ 7H₂O | 1.00 | MgSO₄ 7H₂O | 1.00 |
| CaCl₂ 2H₂O | 0.04 | CaCl₂ 2H₂O | 0.04 |
| CoCl₂ 6H₂O | 0.0080 | CoCl₂ 6H₂O | 0.0080 |
| MnSO₄ H₂O | 0.0200 | MnSO₄ H₂O | 0.0200 |
| CuCl₂ 2H₂O | 0.0020 | CuCl₂ 2H₂O | 0.0020 |
| H₃BO₃ | 0.0010 | H₃BO₃ | 0.0010 |
| Na₂MoO₄ 2H₂O | 0.0004 | Na₂MoO₄ 2H₂O | 0.0004 |
| ZnSO₄ 7H₂O | 0.0040 | ZnSO₄ 7H₂O | 0.0040 |
| Na₂HPO₄ | 2.00 | KH₂PO₄ | 0.70 |
| K₂HPO₄ 3H₂O | 10.48 | K₂HPO₄ 3H₂O | 1.17 |
| (NH₄)₂HPO₄ | 8.00 | NH₄H₂PO₄ | 2.99 |
| (NH₄)₂SO₄ | 5.00 | (NH₄)₂HPO₄ | 3.45 |
| NH₄Cl | 0.13 | (NH₄)₂SO₄ | 8.75 |
| FeSO₄ 7H₂O | 0.04 | NH₄Cl | 0.13 |
| Thiamine | 0.01 | FeSO₄ 7H₂O | 0.04 |
| | | Thiamine | 0.01 |

Table 1 (left): composition of minimal medium MML8AG1 100 (Precultures).
Table 2 (right): composition of minimal medium MML11AG1 100 (Cultures).

Cultures were carried out at 37° C. with an aeration of 1 vvm. The dissolved oxygen was maintained above 30% saturation by controlled shaking (initial speed: 300 rpm; max speed: 1200 rpm) and oxygen was supplied at 0 to 40 ml/min. The pH was adjusted at pH 6.8±0.1 by addition of base (mix of NH4OH 7.5% w/w and NaOH 2.5% w/w). The fermentation was realized in discontinuous fed-batch mode, with a feed stock solution of 700 g/l of glucose. Its composition is showed in table 3.

TABLE 3
composition of the feed stock solution.

| Constituent | Concentration (g/l) |
|---|---|
| Glucose | 700.00 |
| MgSO₄ 7H₂O | 2.00 |
| CoCl₂ 6H₂O | 0.0256 |
| MnSO₄ H₂O | 0.0640 |
| CuCl₂ 2H₂O | 0.0064 |
| H₃BO₃ | 0.0032 |
| Na₂MoO₄ 2H₂O | 0.0013 |
| ZnSO₄ 7H₂O | 0.0128 |
| FeSO₄ 7H₂O | 0.08 |
| Thiamine | 0.01 |

When the glucose ran out in the culture medium, a pulse of fed restored a concentration of 20 g/l of glucose.

After the 5^{th} pulse of fed corresponding to a consumption of 100 g/L of glucose and to a production of about 30 g/L of glycolic acid, the pH of each culture was adjusted at a different pH; pH 7, pH 7.1, pH 7.2, pH 7.3, pH 7.4 or pH 7.6, until the end of the culture. The shift of the pH was done in about 2 hours.

As control, cultures were carried out without any pH modification (final pH 6.7 in table 4).

The cultures were stopped after 40 h of growth at least. Exception was done for the culture with a final pH of 7.6 (culture stopped after 36 h) to avoid a spill over. Production performances of strain AG0662F04c01 grown under these different conditions of final pH are given in the table 4 below. Theses values are given for the highest titer of glycolic acid.

TABLE 4
Impact of the final pH value on the GA
production of the strain AG0662F04c01.

| | | titer [GA] | yield | productivity |
|---|---|---|---|---|
| final pH | trial number | (g/l) | (g GA/g glucose) | (g/l/h) |
| 6.7 | 2 | 45.6 ± 0.3 | 0.28 ± 0.01 | 0.99 ± 0.04 |
| 7.0 | 1 | 50.5 | 0.31 | 1.13 |
| 7.1 | 1 | 52.8 | 0.33 | 1.10 |
| 7.2 | 2 | 55.0 ± 0.1 | 0.33 ± 0.01 | 1.15 ± 0.02 |
| 7.3 | 1 | 56.1 | 0.35 | 1.17 |
| 7.4 | 3 | 53.9 ± 1.6 | 0.36 ± 0.01 | 1.19 ± 0.03 |
| 7.6 | 1 | 35.0 | 0.29 | 1.27 |

To increase the pH after the 5^{th} pulse of fed improves the production of glycolic acid. The yield of production and the titer are much higher when a pH increase is applied to the culture. The best performances were obtained for a final pH ranging from 7.1 to 7.4.

### Example 2

### Process of Fermentation for Producing Glycolic Acid Under Specific pH Conditions

### Impact of the Moment of the pH Increase

In this example, the same shift of pH (from pH 6.7 to pH 7.4) was applied to all the cultures, but at different moment during the fermentation according the amount of glucose consumed (60 g/l to 140 g/l).

The protocol used for this experiment is basically the same as described in Example 1, meaning one step of preculture, and cultures done with the same medium in the same fermenting system.

The pH of each culture was increased from pH 6.7 to pH 7.4 at different moment of the fermentation; after the 3^{rd}, the 4^{th}, the 5^{th}, the 6^{th} or after the 7^{th} pulse of fed meaning after the consumption of respectively 60 g/L, 80 g/L, 100 g/L, 120 g/L, or 140 g/L of glucose. Production performances of strain AG0662F04c01 grown with pH increase at these different moments are given in table below. Theses values are given for the highest titer of glycolic acid.

TABLE 5
Impact of pH increase moment on AG0662F04c01 performances.

| [consumed glucose] | | | yield | |
|---|---|---|---|---|
| at pH increase | trial | titer [GA] | (g GA/g | productivity |
| start (g/l) | number | (g/l) | glucose) | (g/l/h) |
| 60 | 1 | 44.0 | 0.37 | 1.04 |
| 80 | 1 | 45.9 | 0.36 | 1.09 |
| 100 | 3 | 53.9 ± 1.6 | 0.36 ± 0.01 | 1.19 ± 0.03 |
| 120 | 1 | 54.9 | 0.36 | 1.23 |
| 140 | 1 | 57.0 | 0.34 | 1.28 |
| No pH increase | 2 | 45.6 ± 0.3 | 0.28 ± 0.01 | 0.99 ± 0.04 |

The later the pH is increased; higher the titer of glycolic acid produced is. If no shift of pH is applied during the fermentation process, both the yield and the titer are not stabilized and so they are lower than those obtained in condition of pH increase.

### REFERENCES

EP 2 025 759
EP 2 025 760
WO2006/069110 by Dupont de Nemours and Co.
WO2007/141316 by Metabolic Explorer.
A Textbook of Industrial Microbiology, 2^{nd} ed. (1989) Sinauer associates, Sunderland. Md.
Anderson et al., PNAS, 1946, 32, 120-128.
Dechy-Cabaret, O. et al., 2004 Chem. Rev. 104, 6147-6176 Controlled Ring-opening Polymerization of Lactide and Glycolid.
Gerhard et al., Manual of Method of General Bacteriology, Eds P. American Society for Microbiology Washington D.C. (1981).
Hyon, S. -H. et al., 1997 Biomaterials, 18, 1503-1508.
Jahreis et al., J. Bacteriol. 2002, 184 (19) 5307-16.
Miller et al., 1992, A short course in bacterial genetics: a laboratory manual and handbook for Escherichia coli and related bacteria, 2nd Edn. Cold Spring Harbor, N.Y.: Cold Spring Harbor Laboratory.
Michihiko Kataoka, Biosci. Biotechnol. Biochem., 2001.
Penfold and Macaskie, Biotechnol Lett. 2004 December; 26 (24): 1879-83.
Schaefer et al., Anal Biochem. 1999 May 15; 270(1):88-96.
Schmid et al., J. Bacteriol. 1982 July; 151 (1): 68-76.
Sorensen, W. R. & Campbell, T. W, Preparative Methods of Polymer Chemistry 2^{nd} edition, Interscience Publishers Inc, New York 1963.
Tsunekawa et al. Appl Environ Microbiol. 1992 June; 58(6):2081-8.

1-13. (canceled)

14. A method for producing glycolic acid by fermentation, which comprises culturing a microorganism having glycolic acid producing ability in an appropriate culture medium with a carbon source and recovering the glycolic acid from the culture medium, wherein the culture of the microorganism comprises the following steps:
a) culturing the microorganism at a first pH below 7,
b) increasing the pH of the culture medium to a pH above 7, and
c) culturing the microorganism in the culture medium having the increased pH above 7.

15. The method of claim 14, wherein in step a) the pH of the culture medium ranges from 6 to 7 or from 6.5 to 7.

16. The method of claim 14, wherein in step c) the pH of the culture medium ranges from 7 to 8 or from 7.1 to 7.5.

17. The method of claim 14, wherein the step b) is initiated when, in step a), at least one of the following is observed:
the carbon source consumption of the strain is greater than 60 g/L, and/or
the production of glycolic acid is greater than 20 g/L.

18. The method of claim 14, wherein in step b) the pH of the culture medium is increased by addition of a base.

19. The method of claim 18, wherein the base is selected from organic bases or inorganic bases.

20. The method of claim 19, wherein the base is selected from NaOH, NH₄OH, Mg(OH)₂, Ca(OH)₂ or mixtures thereof.

21. The method of claim 18, wherein the culture medium is lacking ammonium cations.

22. The method of claim 14, wherein the microorganism is a microorganism modified for producing glycolic acid and comprises at least one of the following modifications:
attenuation of the genes ldhA and/or mgsA;
attenuation of the gene arcA;
attenuation of the membrane import of glycolate;
attenuation of the conversion of glyoxylate to products other than glycolate;
is unable to substantially metabolize glycolate;
increase of the glyoxylate pathway flux;
increase of the conversion of glyoxylate to glycolate;
increase of the availability of NADPH;
attenuation of the gene aceK; and
combinations thereof.

23. The method of claim 14, wherein the carbon source is at least one of the following: glucose, sucrose, mono- or oligosaccharides, starch or its derivatives or glycerol.

24. The method of claim 14, wherein glycolate is isolated through a step of polymerization to glycolate dimers, oligomers and/or polymers.

25. The method of claim 24, wherein glycolate is recovered by depolymerization from glycolate dimers, oligomers and/or polymers.

26. The method of claim 14, wherein the microorganism is selected from Enterobacteriaceae, Corynebacteriaceae or yeast.

27. The method of claim 22, wherein:
the attenuation of the membrane import of glycolate comprises attenuation of glcA, lldP, and yjcG;
the attenuation of the conversion of glyoxylate to products other than glycolate comprises attenuation of aceB, glcB, gel, eda;
the inability to substantially metabolize glycolate is caused by attenuation of glcDEF, and aldA;
the increase of the glyoxylate pathway flux comprises attenuation of icd, aceK, pta, ack, poxB, iclR or fadR, and/or overexpression of aceA;
the increase of the conversion of glyoxylate to glycolate comprises overexpression of ycdW); and
the increase of the availability of NADPH comprises attenuation of pgi, udhA, edd).
